(19) United States
(12) Patent Application Publication
(10) Pub. No.: US 2004/0176303 A1
(43) Pub. Date: Sep. 9, 2004

(54) Conopeptides and methods of use

(76) Inventors: Frank Mari, Boca Raton, FL (US); Gregg B. Fields, Delray Beach, FL (US)

Correspondence Address: AKERMAN SENTERFITT, P.O. BOX 3188, WEST PALM BEACH, FL, 33402-3188, US

(21) Appl. No.: 10/794,640

(22) Filed: Mar. 5, 2004

Related U.S. Application Data
(60) Provisional application No. 60/452,030, filed on Mar. 5, 2003.

(51) Int. Cl. A61K 38/08; C07K 7/06

(52) U.S. Cl. 514/16; 530/329

(57) ABSTRACT

Disclosed are methods and compositions for modulation of ion levels in cells. The compositions contain a new class of biologically active peptides derived from the venom of predatory marine snails of the species Conus gladiator and Conus mus. The active peptides, termed γ-Hydroxyconophans are heavily hydroxylated small peptides. These peptides contain a definitive structural motif which is a double modification of the polypeptide chain in contiguous residues, i.e., γ-OH-Hyv-D-Trp.

### CROSS REFERENCE TO RELATED APPLICATIONS

The present application claims the priority of U.S. provisional patent application No. 60/452,030, entitled “Therapeutic Agents Containing Gamma Hydroxylated Amino Acids,” filed Mar. 5, 2003. The foregoing is incorporated herein by reference in its entirety.

### STATEMENT AS TO FEDERALLY SPONSORED RESEARCH

This invention was made with U.S. government support under grant numbers GM066004 and CA77402 awarded by the National Institutes of Health. The U.S. government may have certain rights in the invention.

### FIELD OF THE INVENTION

This invention relates generally to the fields of medicine and neuropharmacology. More particularly, the invention relates to a new class of conopeptide compounds useful for binding with specificity to cellular targets such as cell surface receptors.

### BACKGROUND

The venom of predatory marine mollusks belonging to the genus Conus (cone snails) is a rich source of compounds of demonstrated and potential therapeutic use. The Conus venom is a complex mixture of peptides (conopeptides) that elicit a wide range of neurophysiological responses. The venom is used by this marine organism to paralyze its prey. Venom is produced and released by means of a complex venom apparatus including a tubular venom duct wherein the venom is produced by epithelial cells and a muscular bulb used to propel the venom. The bulb is connected to a specialized radular tooth that is used as a harpoon to both impale the prey and reel it in.

Several conopeptides have been shown to be valuable therapeutic agents for the treatment of a variety of neurologically related conditions. The polypeptide components of the venom are produced on ribosomes as protein precursors that subsequently undergo post-translational modifications. The active conopeptides are produced by proteolytic cleavage of the protein precursors. Several classes of conopeptides have been described which exhibit exquisite specificity towards specific neuronal targets such as numerous subclasses of acetylcholine receptors, subtypes of ion channels including sodium, potassium and calcium channels, glutamate receptors, N-Methyl-D-Aspartate (NMDA) receptors and neurotensin receptors. A calcium channel blocking drug based on \acute{ω}-conotoxin MVIIA is presently on the market as a treatment for chronic pain. Other conopeptide-based compounds are thought to be useful in the treatment of Alzheimer's disease, immune system regulation, control of angiogenesis and arrest of malignant growth (Tsetlin, V. I. and Hucho, B., FEBS Lett. 557:9-13, 2004).

There exists a vast number of known species in the genus Conus. Venom from only a fraction of these has been analyzed for the presence of potentially useful conopeptides. Furthermore, the number of distinct conopeptides within any given Conus venom has been shown to be very large. Given the proven usefulness of those few conopeptides presently characterized, there exists great potential to tap this natural combinatorial peptide library as a source for therapeutically useful new compounds.

### SUMMARY

The invention relates to the isolation, synthesis and therapeutic use of compounds and related compositions based on a new class of Conus conopeptides containing the modified amino acid γ-hydroxyvaline (Hyv=V*). These isolated peptides are the first known example of a naturally occurring polypeptide chain containing Hyv. The peptides contain a unique structural motif which is a double modification of the polypeptide chain in contiguous residues, i.e., γ-OH-Hyv-Trp. Yet a further unusual feature of some of the peptides is the fact, that the Trp is in the form of D-Trp. The presence of the γ-OH-Hyv-D-Trp motif in the peptides defines a new class of conopeptides designated herein as γ-Hydroxyconophans. γ-Hydroxyconophans may be defined as containing an amino acid sequence of the general formula H₃CC(O)-Hyv-D-Trp-NH₂. In comparison with known classes of conopeptides, the γ-Hydroxyconophans are particularly a typical because (i) they are not three-dimensionally constrained, in marked contrast to most conopeptides; (ii) they have a high content of hydroxylated residues; iii) they can be unusually short, some embodiments being about eight amino acids in length; and (iv) their primary amino acid sequences have no match with other known peptides in any sequence database.

The amino acid sequences of members of this new class of peptides are based on the discovery of unique octapeptide sequences in the venom of predatory cone snails of the species Conus gladiator and Conus mus. Using a combination of high-resolution analytical methods including nano-NMR and tandem mass spectrometry (MS/MS), the primary sequences, post-translational modifications, chirality and three-dimensional structures of several native conopeptides were elucidated.

Peptides designated herein as gla-1/gla-1′ have the backbone amino acid sequence: A-P-A-N-S-V-W-S (SEQ ID NO:2). Those designated mus-1/mus-1′ have the backbone amino acid sequence: A-P-S-N-S-V-W-S (SEQ ID NO:3). Even without consideration of the unusual modifications discovered to be present in the native conopeptides (i.e., γ-hydroxyproline at residue 2, γ-hydroxyvaline at residue 6 and D-tryptophan at residue 7), the primary structures of the octapeptides represented by SEQ ID NOS:2 and 3 do not match any contiguous sequence of eight amino acids in known or hypothetical proteins described in any database.

Although functional studies using compounds based on γ-Hydroxyconophan sequences in in vitro assays demonstrated a modulatory effect of these peptides on flux of Ca⁺⁺ ions in primary cultures of neurons, consistent with selective binding to target receptors on the cell surfaces, it is likely that specific embodiments of the conopeptides will influence the flux of various cellular ions including sodium, potassium, and chlorine. Therapeutic agents based on these novel sequences and modifications of the amino acid residues therein are likely to find use in a wide variety of applications in which cellular receptors are to be targeted for localization and/or modulation of downstream effects mediated by the receptors.

Accordingly, one aspect of the invention includes an isolated γ-Hydroxyconophan peptide. The peptide includes the amino acids γ-OH-Val and D-Trp in contiguous residues. Other embodiments of the peptide include the amino acid sequence shown herein as SEQ ID NO:2. This peptide is based on the primary sequence of native gla-1 peptide discovered and purified from the venom of Conus gladiator. In another embodiment, the peptide includes the amino acid sequence of SEQ ID NO:3. The latter peptide is based on the corresponding primary sequence (SEQ ID NO:3) found in the mus-1/mus-1′ octapeptide discovered and purified from the venom of Conus mus.

Yet other embodiments of the isolated peptides contain modified amino acids and include an amino acid sequence of the following general structure: A-O-X₁-N-S-X₂-W-S (SEQ ID NO:1) wherein:

O is γ-γ-hydroxyproline;

X₁ is A or S;

X₂ is V or γ-hydroxyvaline (V*); and

W is D-Tryptophan.

Preferred embodiments of the A-O-X₁-N-S-X₂-W-S peptides include peptides having the amino acid sequences shown herein as SEQ ID NOS:5-8. The latter sequences correspond, respectively, to those found in native gla-1, native mus-1 (SEQ ID NOS:5 and 6), and their presumed precursors gla-2 and mus-2 (SEQ ID NOS:7 and 8).

The peptides of the invention can be isolated from an animal, such as a species of Conus, or the peptides can be synthesized by man. The invention further provides pharmaceutical compositions including the isolated peptides.

In another aspect, the invention provides a method of modulating the level of an ion within a cell using the peptides of the invention. The method includes the steps of: (a) providing a cell that responds to a peptide that binds to a chemical structure on the surface of the cell by modulating the level of at least one ion within the cell; and (b) contacting the cell with a peptide including the amino acids γ-OH-Val and D-Trp in contiguous residues, wherein the peptide selectively binds to the chemical structure.

In some embodiments, the chemical structure on the surface of the cell is a cell surface receptor. In preferred embodiments, the receptor is of a selected type, including a calcium channel, a sodium channel, a potassium channel, and a chloride channel. In preferred embodiments, the ion can be a calcium ion, a sodium ion, a potassium ion or a choride ion. In some embodiments, the peptide binds to a receptor that is voltage-gated.

In some embodiments, the peptide used in the method includes the amino acid sequence of SEQ ID NO:2 or SEQ ID NO:3. In other embodiments, the peptide includes an amino acid sequence that may be described as A-O-X₁-N-S-X₂-W-S (SEQ ID NO:1) wherein:

O is γ-γ-hydroxyproline;

X₁ is A or S;

X₂ is V or γ-hydroxyvaline (V*); and

W is D-Tryptophan.

In some embodiments, the method may be practiced using octapeptides having amino acid sequences based on native gla and mus peptides, listed herein as SEQ ID NOS:5-8.

Unless otherwise defined, all technical terms used herein have the same meaning as commonly understood by one of ordinary skill in the art to which this invention belongs.

As used herein, “γ-Hydroxyconophan” is used to describe a new class of conopeptide (i.e., a peptide derived from a Conus species) which is defined as containing an amino acid sequence of the general formula H₃CC(O)-Hyv-D-Trp-NH₂. A “Conophan” is a presumed precursor of a γ-Hydroxyconophan, in which the valine is not hydroxylated, being defined therefore by the general formula H₃CC(O)-V-D-Trp-NH₂.

Although methods and materials similar or equivalent to those described herein can be used in the practice or testing of the present invention, suitable methods and materials are described below. All publications, patent applications, patents, and other references mentioned herein are incorporated by reference for the proposition cited.

### BRIEF DESCRIPTION OF THE DRAWINGS

FIG. 1A is three graphs showing gla conopeptide isolation from the venom of Conus gladiator. Upper and middle plots show fractionation of venom extract using Size Exclusion HPLC (SE-HPLC). The SE-HPLC elution profiles are shown at λ=220 (top) and 280 nm (middle). The arrow indicates the selected fractions of Trp-containing gla peptides. The lower plot shows further fractionation of the major SE-HPLC peaks by Reversed Phase HPLC (RP-HPLC). Three peaks corresponding to peptides gla-1, gla-1′ and gla-2 are seen.

FIG. 1B is three graphs showing mus conopeptide isolation from the venom of Conus mus. Arrow indicates the selected fractions of Trp-containing mus peptides. Isolation steps are as described for FIG. 1A. By RP-HPLC (lower plot), peptides mus-1, mus-1′ and mus-2 are purified in three peaks.

FIG. 2A is a NMR 1D proton spectrum (upper graph) and the corresponding ¹H-NMR 2D-TOCSY spectrum (lower image) of gla-1 peptide purified from C. gladiator. The 2D-TOCSY data were recorded at 25° C. using a gHX HR-MAS probe. The NMR assignments of the γ-hydroxyvaline (i.e., HN: d 7.99, 8 Hz; αH: 4.45, m; γCH₂.m 3.15; βCH m 1.89, 6.9 Hz; γCH₃: d 0.52, 7.1 Hz) correlate well with reported values for synthetic γ-hydroxyvaline.

FIG. 2B is a NMR 1D proton spectrum (upper graph) and the corresponding 2D-TOCSY spectrum (lower image) of gla-1′ peptide purified from C. gladiator. Data were recorded as described for FIG. 2A.

FIG. 2C. is a graph showing a NMR 2D-TOCSY spectrum of native gla-2 peptide isolated from the venom of C. gladiator. Data were recorded as described for FIG. 2A.

FIG. 3A is a graph showing mass spectrometry (ESI-MS/MS) spectrum of the gla-1 peptide from C. gladiator.

FIG. 3B is a graph showing ESI-MS/MS spectrum of the gla-1′ peptide from C. gladiator.

FIG. 4A is two graphs showing NMR 1D proton spectra of conopeptides mus-1 (upper plot) and mus-1′ (lower plot) purified from C. mus.

FIG. 4B is a graph showing ESI-MS/MS spectrum of the mus-1 peptide from C. mus.

FIG. 4C is a graph showing ESI-MS/MS spectrum of the mus-1 peptide from C. mus.

FIG. 5 is three graphs showing a comparison of NMR spectra (1D proton spectrum, 0.2-4.5 ppm region, αH & side chains) of native gla-2 conophan (upper) and corresponding synthetic peptides of gla-2 containing either L-Trp (middle) and D-Trp (lower). The arrows in the upper and lower graphs indicate resonances that are dissimilar in synthetic gla-2 containing L-Trp (middle) compared with native gla-2 (upper) or synthetic gla-2 with D-Trp (lower), confirming the chirality assignment of Trp-7 in gla-2.

FIG. 6A is a graph showing ESI-MS/MS of the native gla-2 conophan from C. gladiator. Spectra were recorded using a LCQ-Deca Ion trap instrument.

FIG. 6B is a graph showing ESI-MS/MS of synthetic gla-2 containing L-Trp.

FIG. 6C is a graph showing ESI-MS/MS of synthetic gla-2 containing D-Trp.

FIG. 7A is a graph showing chromatographic characterization of two synthetic analogs of the gla-1 γ-Hydroxyconophan, i.e., gla-1(Hyv6Thr′) and gla-1(Hyv6Thr′). The graph shows a RP-HPLC profile of the two analogs using the chromatographic conditions used in FIG. 1. The difference in retention of these analogs is the same as the gla-1/gla-1′ pair.

FIG. 7B is a graph showing ESI-MS/MS spectral characterization of gla-1 synthetic analog gla-1(Hyv6Thr).

FIG. 7C is a graph showing ESI-MS/MS spectral characterization of gla-1 synthetic analog gla-1(Hyv6Thr′). Comparing FIGS. 7B and 7C with FIGS. 3A and 3B (native gla-1 and gla-1′, respectively), it is seen that the ESI-MS/MS spectra of the two analogs of gla-1 have similar fragmentation patterns to those of the native peptides.

FIG. 8 shows the chemical structure of the native gla-1 octapeptide.

FIG. 9 is a drawing depicting a molecular model of a γ-Hydroxyconophan structural motif H₃CC(O)-Hyv-D-Trp-NH₂. This structure illustrates the proximity of the γ-methyl of Hyv to D-Trp. Evidence of this interaction is provided by the strongly shielded chemical shift of this methyl group shown in FIGS. 3 and 4. D-Trp provides steric impediment to the lactonization process (represented by the electron clouds of the side chains of these modified amino acids), thereby providing stability to polypeptide chains that include this structural motif.

### DETAILED DESCRIPTION

The invention encompasses compositions and methods relating to a novel class of biologically active conopeptides, termed “Conophans, and “γ-Hydroxyconophans” which can be obtained by purification from the venom of predatory cone snails of the genus Conus. Alternatively, the compositions can be synthesized. The below described preferred embodiments illustrate various compositions and methods within the invention. Nonetheless, from the description of these embodiments, other aspects of the invention can be made and/or practiced based on the description provided below.

### Biological Methods

Methods involving conventional and analytical chemistry, molecular biological and cell biological techniques are described herein. Such techniques are generally known in the art and are described in detail in methodology treatises such as Classics in Total Synthesis. Targets, Strategies, Methods, K. C. Nicolaou and E. J. Sorensen, VCH, New York, 1996; The Logic of Chemical Synthesis, E. J. Coney and Xue-Min Cheng, Wiley & Sons, NY, 1989; and NMR of Proteins and Nucleic Acids, Wuthrich, K., Wiley & Sons, New York, 1986. Molecular biological and cell biological methods are described in treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Methods involving conventional biology and neurobiology and neuropharmacology are also described herein. Such techniques are generally known in the art and are described in methodology treatises such as Sambrook et al., supr. Conopeptides and related toxins are generally reviewed, for example, in Tsetlin, V. I. and Hucho, B., FEBS Lett. 557:9-13, 2004, and in Mari, F. and Fields, G. B, Chimica Oggi 21:23-48, 2003.

### Conopeptides Having Novel Amino Acid Sequences

The invention relates to a novel class of conopeptides having several features that set them apart from all other previously described classes of conopeptides. As described in detail in the Examples below, these peptides were isolated and characterized from the venom of cone snails of the species Conus gladiator and Conus mus. Using a combination of protein purification and analytical techniques, a novel class of conopeptides was identified having the following general structure:

A-O-X₁-N-S-X₂-W-S (SEQ ID NO:1) wherein:

O is γ-hydroxyproline;

X₁ is A or S;

X₂ is V or γ-hydroxyvaline (V*); and

W is D-Tryptophan

This general structure defines a motif that was conserved in native octapeptides isolated from two evolutionarily diverse species that inhabit different geographic regions, i.e., Conus gladiator and Conus mus. The structure of these peptides is distinguished by the presence of unusual amino acid modifications, i.e, hydroxyproline in residue 2 and D-Trp in residue 7. Within this novel class of peptides are found two subclasses of native octapeptides which differ between the two species by only one amino acid, i.e., at residue 2 (X₁). This variation at residue 2 is a conservative change from an Alanine in the gla peptide subclass to a Serine in the mus subclass of peptides. Additionally, peptides which are believed to be the mature, most active forms include hydroxyvaline in residue 6 (X₂), whereas their presumed precursors contain unhydroxylated valine at this position.

Even without their unusual post-translational modifications, the native gla and mus conophan peptides both exhibit primary sequence structures not found in any known or hypothetical protein or peptide, as shown below:

gla-1/gla-1′-gla-2: A-P-A-N-S-V-W-S (SEQ ID NO:2)

mus-1/mus-1′/mus-2: A-P-S-N-S-V-W-S (SEQ ID NO:3)

Conservation of the highly similar amino acid backbone in the two subclasses of octa-conopeptides suggests that this backbone is likely to form an important scaffold for preservation of the function of these molecules. As used herein, the term “backbone amino acid sequence” refers to the primary structure of the peptide prior to post-translational modification.

### Conopeptides Containing Hydroxylated Amino Acids

Many preferred embodiments of the peptides contain post-translational modifications that were discovered in the native conopeptides. The name given to one of the new classes of compounds, i.e., the γ-Hydroxyconophans, is derived in part from the finding of a post-translation modification of hydroxylation of valine in the gamma position in native gla-1 and mus-1 peptides. (Conophans, exemplified by gla-2 and mus-2, by contrast, lack the hydroxylation on the valine residue, and are thought to be the precursors of gla-1 and mus-1, respectively, which are acted upon by a presumed valine hydroxylase enzyme to form the mature γ-Hydroxyconophans.) In the γ-Hydroxyconophan class of conopeptides, the residue in position 6 is γ-Hydroxyvaline (γ-Hyv; V*).

Based on studies described in Examples below, the stability of γ-Hyv within these conopeptides is thought to be preserved by the presence of D-Trp as the adjacent residue. This dyad of modified amino acids represents a novel structural motif that characterizes the γ-Hydroxyconophan class of conopeptides. Accordingly, the γ-Hydroxyconophans can be described by the general structural formula:

A-O-X₁-N-S-V*-W-S (SEQ ID NO:4)

wherein:

O=γ-hydroxyproline;

V*=γ-hydroxyvaline; and

W=D-Trp.

Naturally occurring members of this class include the following octapeptides:

gla-1/gla-1′: A-O-A-N-S-V*-W-S (SEQ ID NO: 5)

mus-1/mus-1′ A-O-S-N-S-V*-W-S (SEQ ID NO: 6)

wherein:

O=γ-hydroxyproline;

V*=γ-hydroxyvaline; and

W=D-Trp.

Prior to the demonstration herein, γ-Hyv had not been found within a polypeptide chain (Hernandez, I. L. C., Godinho, M. J. L., Magalhaes, A., Schefer, A. B., Ferreira, A. G., Berlinck, R. G. S. Journal of Natural Products 2000, 63, 664-665; Krishna, R. G., Wold, F. In Proteins: Design and Analysis; Angeletti, R. H., Ed.; Academic Press: San Diego, Calif., 1998, pp 121-206). This is not surprising for the following reason. The hydroxylation of valine would be an unexpected post-translational modification in proteins and peptides because the hydroxyl group could readily cleave the peptide bond by intraresidue cyclization to form a lactone. Applicants, not seeking to be bound to this or any other theory, present a mechanism for the existence of γ-Hyv in the γ-Hydroxyconophan octopeptide relating to its proximity to the contiguous D-Trp in residue 7.

The presence of hydroxlated amino acids (i.e., in position 2 and position 6) is a prominent feature of the γ-Hydroxyconophans and is believed to impart structural stability to these molecules. Additionally, it is generally agreed that the hydroxylation which is observed in many classes of known conopeptides may be related to hydrogen bonding directed towards increasing binding strength and selectivity towards their neuronal targets, which are typically cell surface receptors. Although preferred embodiments of the peptides are described as having hydroxylated proline and valine, hydroxylated amino acids in general are known as an important class of modified amino acids in proteins. For example, γ-Hydroxyproline (γ-Hyp) and δ-hydroxylysine (δ-Hyl) are commonly found in collagen and are vital for collagen structural stability (Haading, J. J., Crabbe, M. J. C. Post-translational Modifications of Proteins, CRC Press: Boca Raton, Fla., 1992; Perret, S., Merle, C., Bernocco, S., Berland, P., Garrone, R., Hulmes, D. J. S., Theisen, M., Ruggiero, F. J. Biol. Chem. 2001, 276, 43693-43698). γ-Hydroxyarginine has been found as part of the sequence of polyphenolic proteins that form the adhesive plaques of marine mussel species (Papov, V. V., Diamond, T. V., Biemann, K, Waite, J. H. J. Biol. Chem. 1995, 270, 20183-20192). Accordingly, those of skill in the art will appreciate that peptides based on the disclosed amino acid backbones and post-translation modifications could readily be envisoned and produced by substitution with other hydroxylated amino acids without departing from the spirit and scope of the invention.

### Synthesis of Conophan and γ-Hydroxyconophan Conopeptides

As shown in Examples below, the peptides of the invention can be synthesized starting from amino acids by methods well known in the art. Alternatively, it will be readily apparent to those of skill in the art of molecular biology that cDNA molecules can be isolated that encode the venom proteins (that are subsequently cleaved to form the active conopeptides), starting with PCR primers based on the disclosed amino acid sequences of the peptides. Using well known methods, the cDNAs can be used, for example, to transfect cells for in vitro or in vivo production of recombinant proteins and peptides.

The peptides of the invention can vary in length, but are preferably between about 5 and 200 amino acids in length, more preferably between about 5 and 50 amino acids in length, and most preferably between about 5 and 25 amino acids in length. The peptides can be modified using many methods standard in the art depending upon their intended use.

### Utility of γ-Hydroxyconophans and Conophans

As members of a new class of conopeptides, the γ-Hydroxyconophans and Conophans can be used as agents that selectively-bind to and thereby modulate the function of specific cell surface receptors on cells, for example on neurons. Various classes of conopeptides are known to selectively bind to cellular receptors including numerous subclasses of acetylcholine receptors, subtypes of ion channels including sodium, potassium, calcium and chloride channels, glutamate receptors, N-Methyl-D-Aspartate (NMDA) receptors and neurotensin receptors. As shown in Examples below, gla-1 and gla-2 peptides derived from the octapeptide sequences of gla-1 and gla-2 were shown to modulate Ca⁺⁺ flux in cultured neurons from rat cortex. This effect was presumed to be mediated through cell surface receptors that either directly or indirectly control calcium ion concentration in the cells.

Accordingly, in one aspect the invention provides a method for modulating the level of an ion within a cell. The method includes the steps of: (a) providing a cell that responds to a peptide that binds to a chemical structure on the surface of the cell by modulating the level at least one ion within the cell; and (b) contacting the chemical structure on the surface of said cell with a peptide containing the amino acids γ-OH-Val and D-Trp in contiguous residues, wherein the peptide selectively binds to the chemical structure. An exemplary chemical structure on the cell surface is a receptor, of which many different types are known, as described above, including inter alia calcium channels, sodium channels, potassium channels and chloride channels. Selective binding of particular classes of receptors by the peptides of the invention can be determined empirically by testing the peptides in receptor assay systems that are well established for this purpose.

### Animal Subjects

Because control of ion flux in cells is central to a very large number of physiological processes in animals and man, the invention is believed to be compatible with any animal subject. A non-exhaustive list of examples of such animals includes mammals such as mice, rats, rabbits, goats, sheep, pigs, horses, cattle, dogs, cats, and primates such as monkeys, apes, and human beings. Those animal subjects that have a disease or condition that relates to modulation of calcium levels within a cell are preferred for use in the invention, as these animals may have the symptoms of their disease reduced or even reversed. In particular, human patients suffering from neurologic disorders such as Alzheimer's disease, immune system dysfunction, and cancer might particularly benefit.

### Administration of Compositions

The compositions of the invention may be administered to animals including humans in any suitable formulation. For example, the compositions may be formulated in pharmaceutically acceptable carriers or diluents such as physiological saline or a buffered salt solution. Suitable carriers and diluents can be selected on the basis of mode and route of administration and standard pharmaceutical practice. A description of other exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions.

The compositions of the invention may be administered to animals by any conventional technique. Such administration may be oral or parenteral (for example, by intravenous, subcutaneous, intramuscular, or intraperitoneal introduction). The compositions may also be administered directly to the target site by, for example, surgical delivery to an internal or external target site, or by catheter to a site accessible by a blood vessel. Other methods of delivery, for example, liposomal delivery or diffusion from a device impregnated with the composition, are known in the art. The compositions may be administered in a single bolus, multiple injections, or by continuous infusion (for example, intravenously or by peritoneal dialysis). For parenteral administration, the compositions are preferably formulated in a sterilized pyrogen-free form.

Compositions of the invention can also be administered in vitro to a cell (for example, in in vitro assays to modulate calcium levels within the cells, or to target particular cell surface receptors capable of selectively binding-these peptides).

### Effective Doses

An effective amount is an amount which is capable of producing a desirable result in a treated animal or cell (for example, reduced calcium flux in the cells of the animal or in a cell in culture). As is well known in the medical and veterinary arts, dosage for any one animal depends on many factors, including the particular animal's size, body surface area, age, the particular composition to be administered, time and route of administration, general health, and other drugs being administered concurrently. It is expected that an appropriate dosage for parenteral or oral administration of compositions of the invention would be in the range of about 1 μg to 100 mg/kg of body weight in humans. An effective amount for use with a cell in culture will also vary, but can be readily determined empirically (for example, by adding varying concentrations to the cell and selecting the concentration that best produces the desired result). It is expected that an appropriate concentration would be in the range of about 0.0001-100 mM. More specific dosages can be determined, for example, using a cultured neuronal cell assay as described below.

Toxicity and efficacy of the compositions of the invention can be determined by standard pharmaceutical procedures, using cells in culture and/or experimental animals to determine the LD₅₀ (the dose lethal to 50% of the population) and the ED₅₀ (the dose that effects the desired result in 50% of the population). Compositions that exhibit a large LD₅₀/ED₅₀ ratio are preferred. Although less toxic compositions are generally preferred, more toxic compositions may sometimes be used in in vivo applications if appropriate steps are taken to minimize the toxic side effects.

Data obtained from cell culture and animal studies can be used in estimating an appropriate dose range for use in humans. A preferred dosage range is one that results in circulating concentrations of the composition that cause little or no toxicity. The dosage may vary within this range depending on the form of the composition employed and the method of administration.

### EXAMPLES

The present invention is further illustrated by the following specific examples, which should not be construed as limiting the scope or content of the invention in any way.

### Example 1

### Collection of Conus Specimens and Venom

Specimen collection and venom isolation. Conus gladiator is a predatory worm-hunting cone species that inhabits shallow water along the Pacific coast from the Gulf of California to Peru. Specimens were collected from several locations off the Pacific coast of Costa Rica. The snails were dissected to obtain their venom ducts. Crude venom extraction was performed in H2O/Acetonitrile (40/60)+0.1% TFA. The venom extract was filtered and centrifuged at 10,000 rpm at 5° C. for 10 min. The supernatant was pooled and lyophilized (˜50 mg from 47 snails) and used for subsequent analyses: Specimens of Conus mus were collected off the Florida Keys (Plantation, Monroe County). Crude venom (12 mg) was extracted from these snails and processed as described for Conus gladiator.

### Example 2

### Chromatographic Separation of Conus Venom

Peptide isolation. Initial fractionation of the venom lyophylates was performed using SE-HPLC on a Pharmacia Superdex-30 column (2.5×100 cm) with elution by 0.1 M NH₄HCO₃ buffer at a flow rate of 1.5 ml/min. The column eluent was monitored on a PDA detector (TSP SM-5100) at λ=220 and 280 nm. The Trp-containing fractions in the major peak (gla_{—}09) in the λ=280-detected chromatogram were further separated using an RP-HPLC Vydac C18 column (10×250 mm, 5 μm, 300 Å) eluted with a linear gradient of H₂O/60% CH₃CN over 100 min. TFA (0.1%) was used as ion-pairing reagent.

### Example 3

### 1D and 2D NMR Spectroscopy of Conus Peptides

NMR spectra were acquired on a Varian Inova 500 MHz spectrometer (Varian, Palo Alto, Calif.) equipped with three RF channels, pulse field gradients and waveform generators. Initially, 1D and 2D-TOCSY spectra were recorded using a gHX HR-MAS probe for 1 nmol of gla-1 in 35 μl. The gla conopeptides were isolated in nanomolar quantities, whereas the mus conopeptides were isolated in picomolar quantities. The use of a High Resolution Magic Angle Spinning (HR-MAS) probe (Barbara, T. M. Journal of Magnetic Resonance Series A 1994, 109, 265-269; Barbara, T. M., Bronnimann, C. E. Journal of Magnetic Resonance 1999, 140, 285-288) along with related NMR techniques (Matei, E., Pflueger, F. C., Franco, A., Cano, H., Mora, D., Mari, F. Paper presented at the 18th APS, 2003) allowed the acquisition of NMR spectra from such limited amounts of material. The larger sample quantities (20-35 nmol of the gla peptides) were analysed using 3 mm sample tubes in 130 μl of NMR solution in a 5 mm gHCX triple resonance probe. 1D spectra were acquired using 512 scans, whereas 2D spectra were acquired using 96 increments in t1 with 256 scans per increment in a phase sensitive mode. 2D spectra were processed using linear predictions in t1 to 1024 points and transformed to final size of 2k×2k. The 1D spectra of picomolar amounts of the mus conopeptides were acquired overnight using 3 mm sample tubes in 130 μl of NMR solution in a 5 mm gHCX triple resonance probe. All spectra were recorded at 25° C. and 0° C in an NMR solution that consisted of 90% H₂O/10% D₂O using TSP as an internal standard. The pH of this solution was adjusted to 3.6 using 0.01 M solutions of HCl and NaOH and a Phoenix micro-pH probe. Water suppression was achieved using WATERGATE (Piotto, M., Saudek, V., Sklenar, V. Journal of Biomolecular Nmr 1992, 2, 661-665) and Excitation Sculpting (Callihan, D., West, J., Kumar, S., Schweitzer, B. I., Logan, T. M. Journal of Magnetic Resonance Series B 1996, 112, 82-85) for the 2D experiments and WET (Smallcombe, S. H., Patt, S. L., Keifer, P. A. Journal of Magnetic Resonance Series A 1995, 117, 295-303) and presaturation for the 1D ¹H spectra. The resonance assignments were carried out using standard biomolecular NMR procedures.

### Example 4

### Analysis of Conus Peptides by Positive Ion Electrospray Ionization Mass Spectrometry (ESI-MS/MS)

Positive ion electrospray ionization mass spectrometry (ESI-MS/MS) spectra of all conopeptides (gla-1 and gla-2) were obtained on either a Micromass Q-TOF micro (Waters Corporation, Milford, Mass.) or a Finningan LCQ-Deca instrument (Finnigan, Germany). Samples analysed (˜1 pmol) using the Q-TOF instrument were desalted using a C18 ZipTip and analyzed using a nanospray ion source (Wilm, M., Shevchenko, A., Houthaeve, T., Breit, S., Schweigerer, L., Fotsis, T., Mann, M. Nature 1996, 379, 466-469; Wilm, M., Mann, M. Analytical Chemistry 1996, 68, 1-8). Glu-fibrinogen, m/z=785.85 doubly charged, was used as an internal standard. Samples containing ˜10 pmol of peptide were used for analyses using the LCQ instrument. Samples were analyzed by flow injection using 30% ACN/0.1% acetic acid as a carrier. Charged states of the peptides were confirmed by the use of the Zoom-Scan method (van Eijk, H. M. H., Rooyakkers, D. R., Soeters, P. B., Deutz, N. E. P. Analytical Biochemistry 1999, 271, 8-17).

### Example 5

### Peptide Synthesis and Sequencing of Conus Peptides by Edman: Degradation

Amino acid sequencing was carried out by Edman degradation chemistry on an ABI Procise cLC instrument. Peptide synthesis was performed on an Applied Biosystems 433A peptide synthesizer (Applied Biosystems, Foster City, Calif.). Peptide-resin cleavage utilized appropriate-scavengers (King, D. S., Fields, C. G., Fields, G. B. International Journal of Peptide and Protein Research 1990, 36, 255-266; Fields, C. G.; Fields, G. B. Tetrahedron Letters 1993, 34, 6661-6664) to avoid Trp oxidation. The cleaved peptides were purified by HPLC as described above.

### Example 6

### Isolation and Chromatographic Separation of gla and mus Peptides from Conus Venom

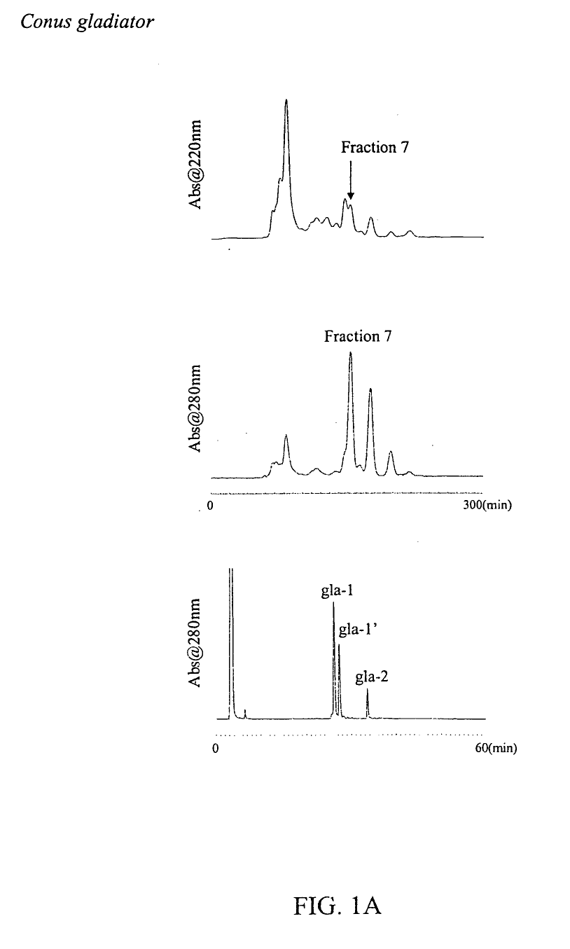

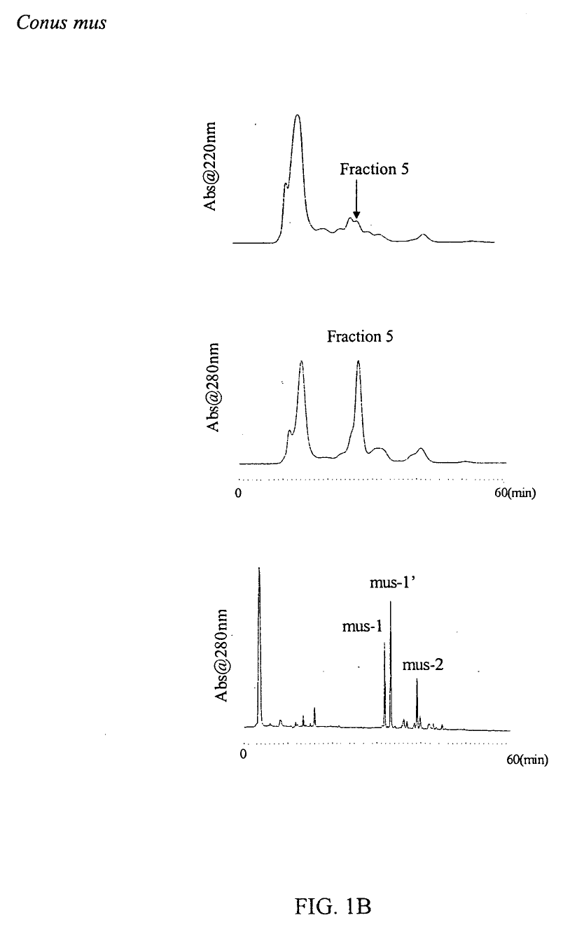

FIG. 1A shows results of fractionation and purification of the venom of Conus gladiator. Further separation of the material in the major peak (gla-09) in the λ=280-detected chromatogram by RP-HPLC yielded three peptide fractions, i.e., gla-1, gla-1′, and gla-2 (FIG. 1A, lower). Similar profiles were obtained by fractionation of the venom of Conus mus (FIG. 1B).

### Example 7

### 1D and 2D NMR Spectroscopy of gla and mus Conopeptides

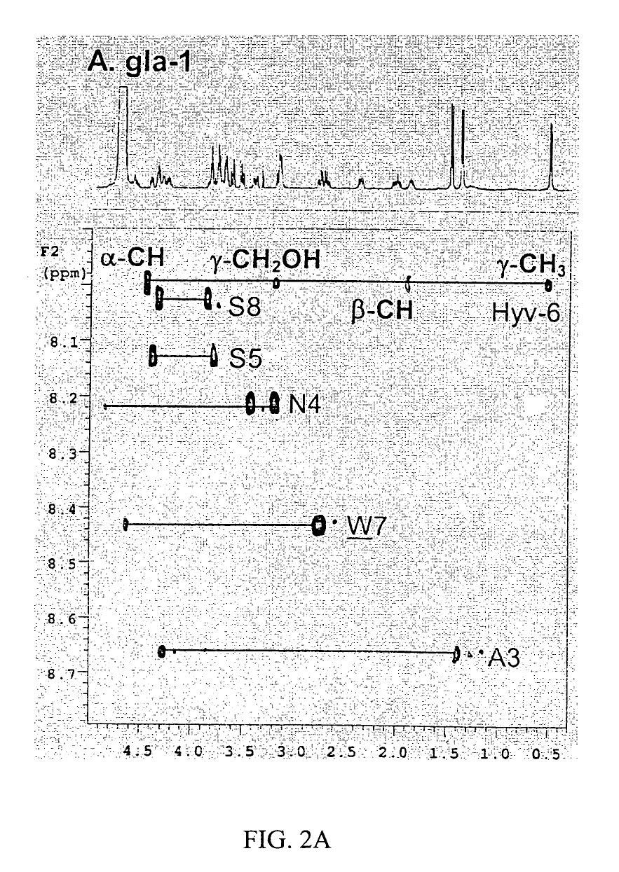

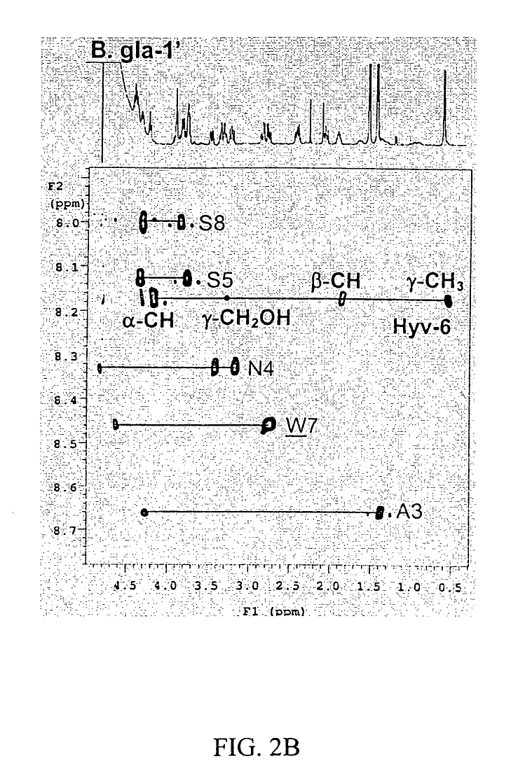

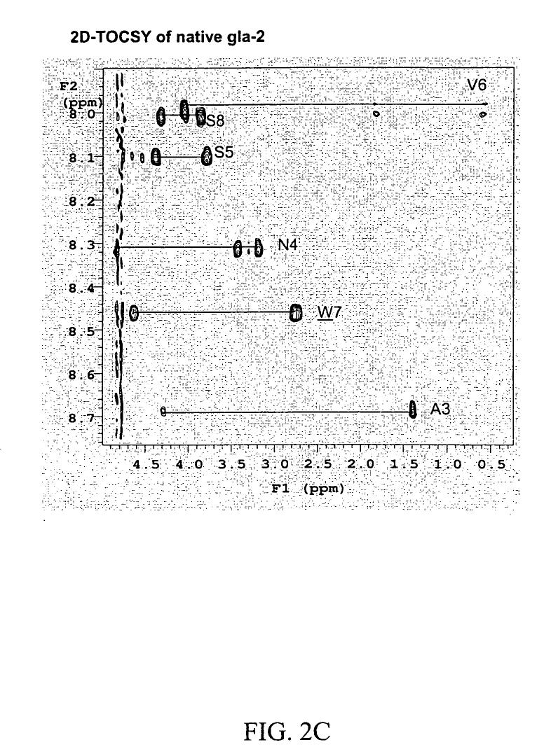

The fractions obtained by RP-HPLC were subsequently analyzed by NMR as described in Example 3 above. Referring to FIGS. 2A-C, the nano-NMR spectra of gla-1 peptides revealed an amino acid composition as follows:

gla-1: 2 Ala, 2 Ser, Trp, Asn, Hyp, and an unusual amino acid (FIG. 2A);

gla-1′: 2 Ala, 2 Ser, Trp, Asn, Hyp, and an unusual amino acid (FIG. 2B); and

gla-2: 2 Ala, 2 Ser, Trp, Asn, Hyp, and Val for gla-2 (FIG. 2C).

### Example 8

### MS/MS Spectra of gla and mus Conopeptides

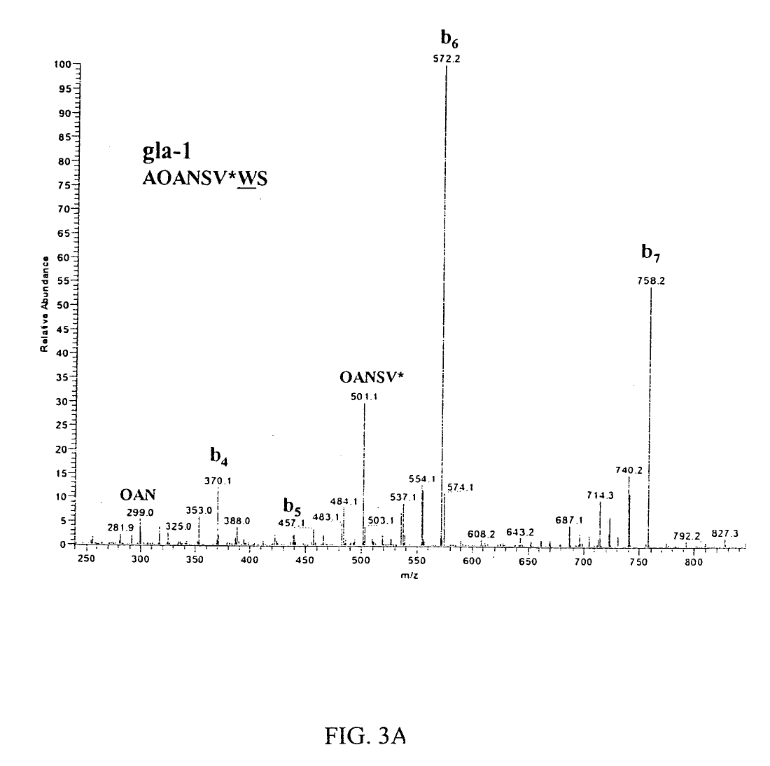

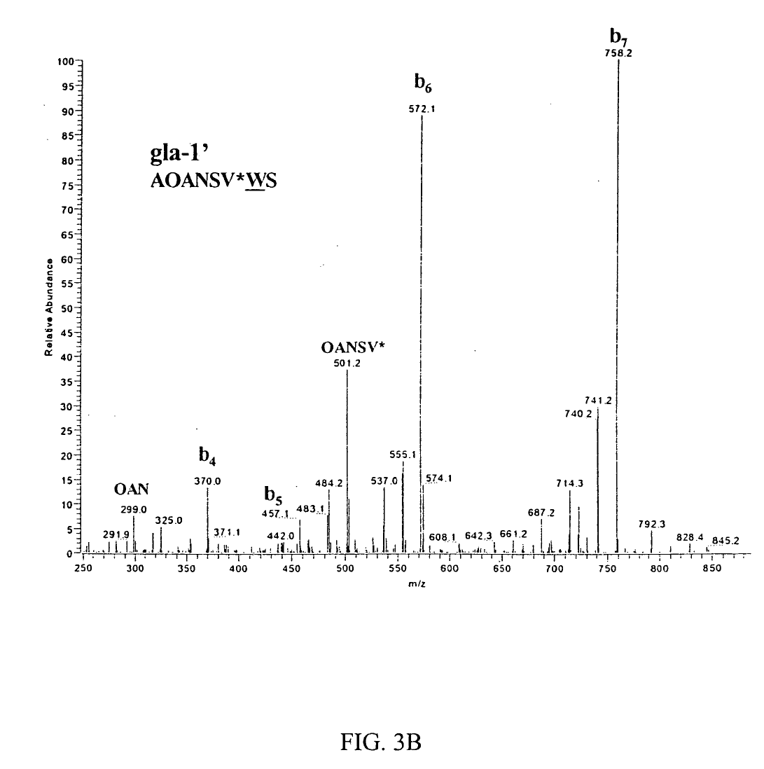

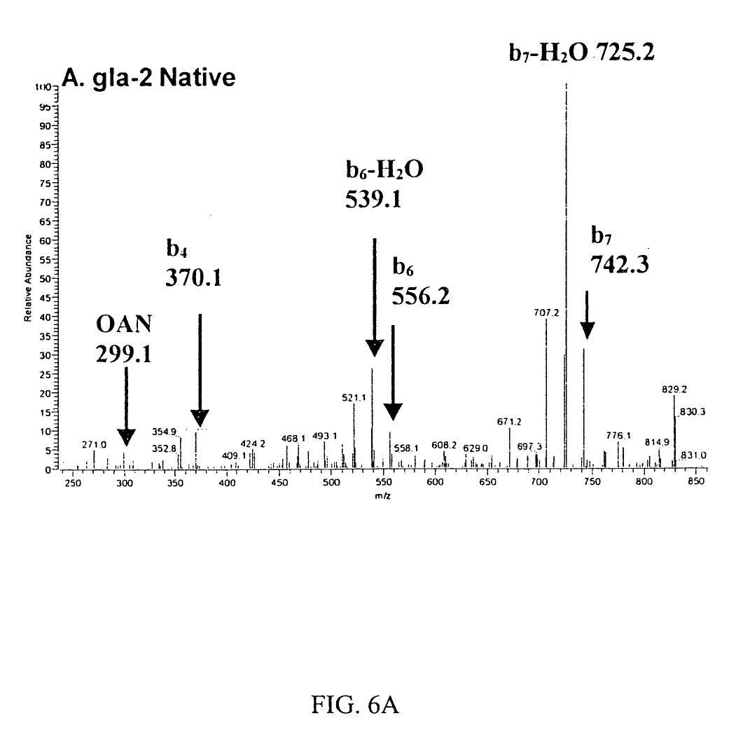

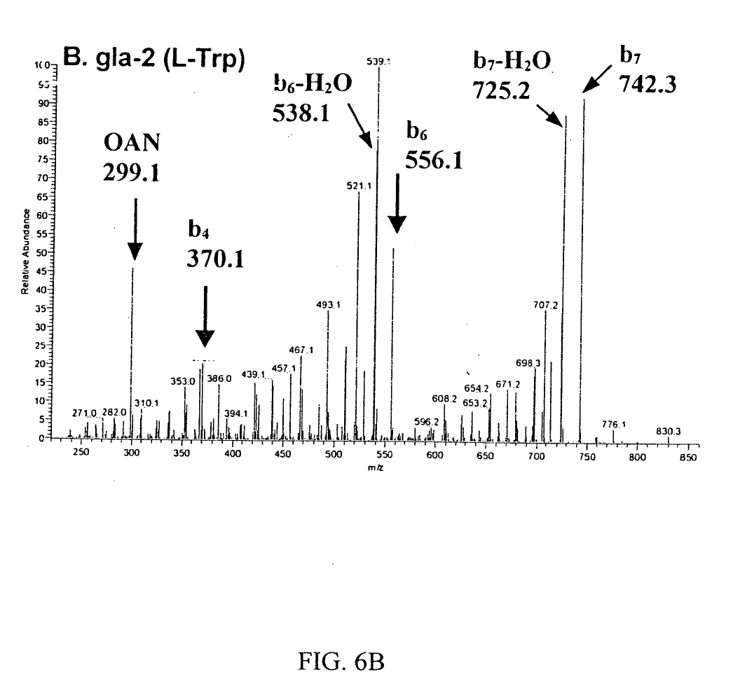

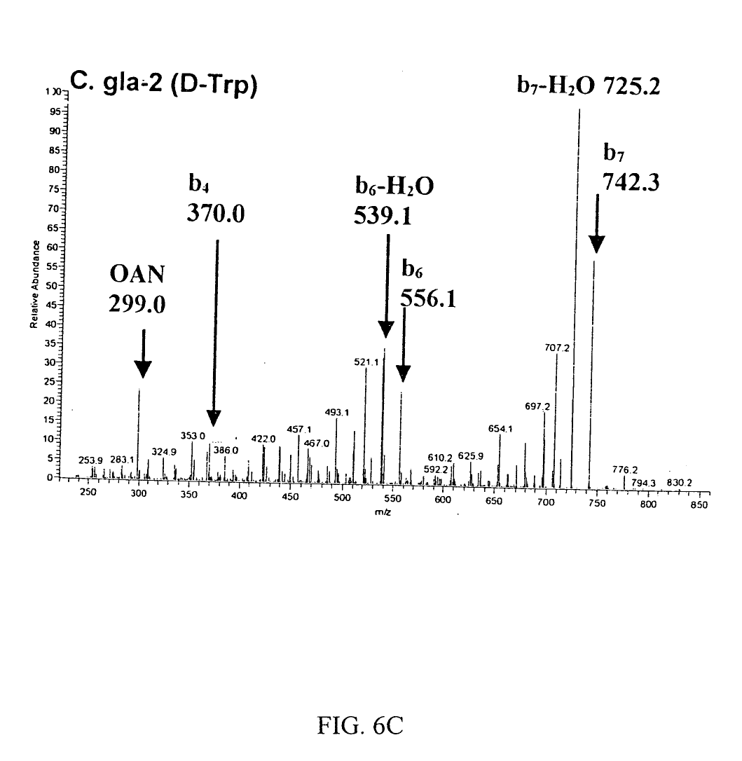

The gla and mus peptides were further analyzed by MS/MS. The mass spectra of gla-1/gla-1′ gave molecular ions of 863.3 Da (FIGS. 3A and 3B). By contrast, the molecular ions of gla-2 were 847.3 Da (FIG. 6A). The NMR and MS data were consistent with gla-1 and gla-1′ having the same covalent structures.

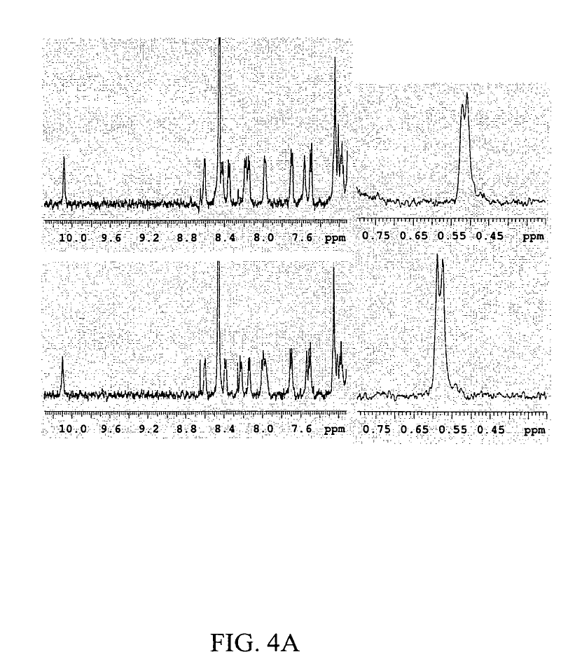

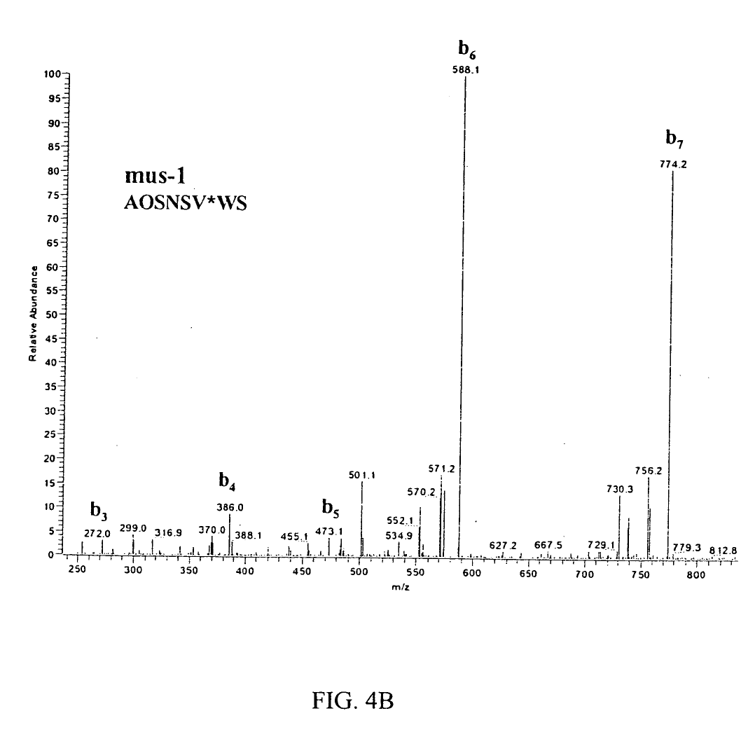

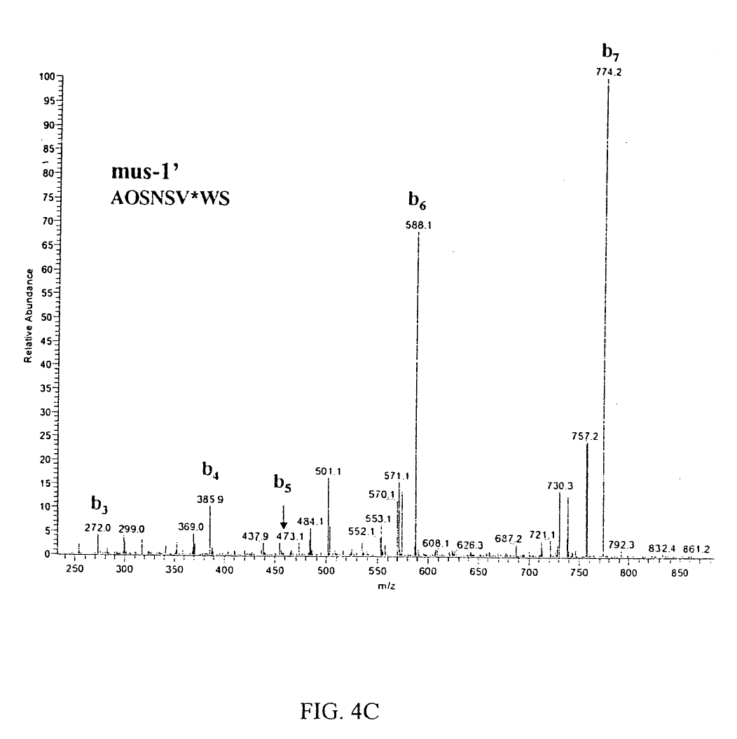

FIGS. 4A-C show corresponding NMR and MS/MS data for the mus octapeptides. The analysis revealed molecular ions of 879.3 Da for the mus-1/mus-1′ pair (FIGS. 4B, 4C) and 863.3 Da for the mus-2 octapeptide.

MS-based analysis revealed the following an amino acid compositions for the mus octapeptides:

mus-1: 3 Ser, Ala, Trp, Asn, Hyp, and an unusual amino acid;

mus-1: 3 Ser, Ala, Trp, Asn, Hyp, and an unusual amino acid; and

mus-2: 3 Ser, Ala, Trp, Asn, Hyp, and Val.

### Example 9

### Edman Degradation Analysis of gla and mus Conopeptides

Edman degradation sequence analysis of gla-1 resulted in Ala-Hyp-Ala-Asn-Ser, which is smaller than indicated by the NMR and MS results. By way of contrast, gla-2 was sequenced to completion by Edman degradation chemistry, resulting in a primary structure consistent with the NMR and MS/MS data.

### Example 10

### Synthetic Peptides and Confirmation of Presence of D-Trp in γ-Hydroxyconophans

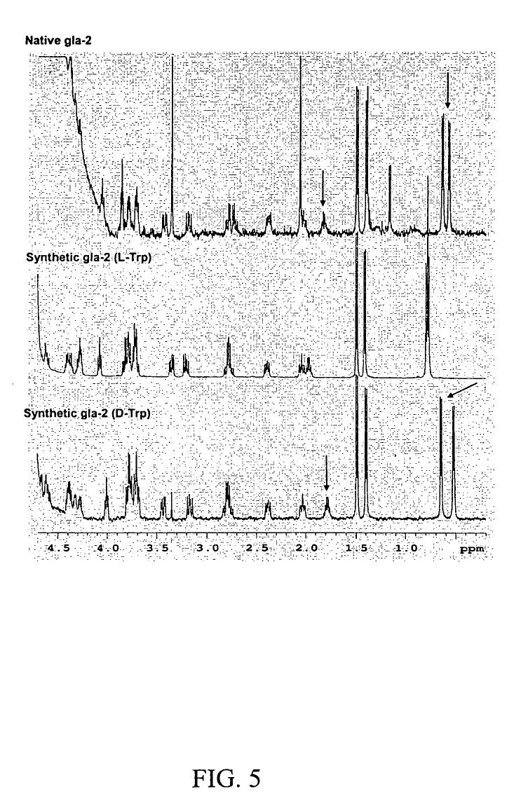

Synthetic γ-Hydroxyconophans corresponding to the gla and mus peptides were synthesized as described in Example 5. To confirm the presence of D-Trp in position 7 of the naturally occurring octapeptides, synthetic gla-2 peptides were constructed having either D-Trp or L-Trp in position 7. FIG. 5 shows 1D NMR chromatographic profiles of native gla-2, and the two synthetic peptides. FIG. 6 (A-C) shows a comparison of these peptides by MS/MS. The stereochemistry of the tryptophan residue was determined by comparing the NMR (FIG. 5) and MS/MS spectra of the native gla-2 with its synthetic peptide analogs (FIGS. 6A-C). The strong influence of the chirality of Trp-7 on the MS/MS fragmentation pattern of these peptides was central for the determination.

Results from both of these analyses demonstrated behavior of native gla-2 that was consistent with that of the synthetic sample containing D-Trp. This result thus confirmed the presence of D-Trp in the γ-Hydroxyconophan sequence.

### Example 11

### Characterization of Hydroxyvaline (Hyv₆) Diastereomers of gla and mus Conopeptides

As described above, the gla-1/gla-1′ and mus-1/mus-1′ pairs were shown to have the same respective covalent structures. Nevertheless, their chromatographic behaviors revealed differences in hydrophobicity in a temperature-independent fashion. In principle, these differences might be attributed to cisltrans isomerism of the peptide bond involving Hyp in residue 2, as suggested by NMR evidence in other related conopeptides (Pallaghy, P. K., He, W., Jimenez, E. C., Olivera, B. M., Norton, R. S. Biochemistry 2000, 39, 12845-12852). By contrast, ultraviolet resonance Raman spectroscopy suggested that two conformational states within conopeptides could be attributed to the differences of the χ dihedral angles of the D-Trp within their sequence (Jimenez, E. C., Watkins, M., Juszczak, L. J., Cruz, L. J., Olivera, B. M. Toxicon 2001, 39, 803-808). However, in these cases, temperature dependency of the distribution of the conformers has been observed (Jacobsen, R., Jimenez, E. C., Grilley, M., Watkins, M., Hillyard, D, Cruz, L. J., Olivera, B. M. Journal of Peptide Research 1998, 51, 173-179).

To determine the explanation for the differing chromatographic profiles in the case of the gla and mus conopeptide pairs, a detailed analysis of the MS/MS data of the gla and mus γ-Hydroxyconophans was undertaken. The result revealed that the fragmentation patterns within the pairs differs in the intensity of the b₆ fragment (Compare FIGS. 3A, 3B and 6A), suggesting structural differences within the Hyv residue. Furthermore, it was noted that the largest chemical shift differences between gla-1 and gla-1′ are within the resonances of Hyv (FIG. 2). Hyv bears a chiral center at the β carbon; therefore, gla-1 and gla-1′ were postulated to be diastereomers, epimeric at the β carbon of Hyv.

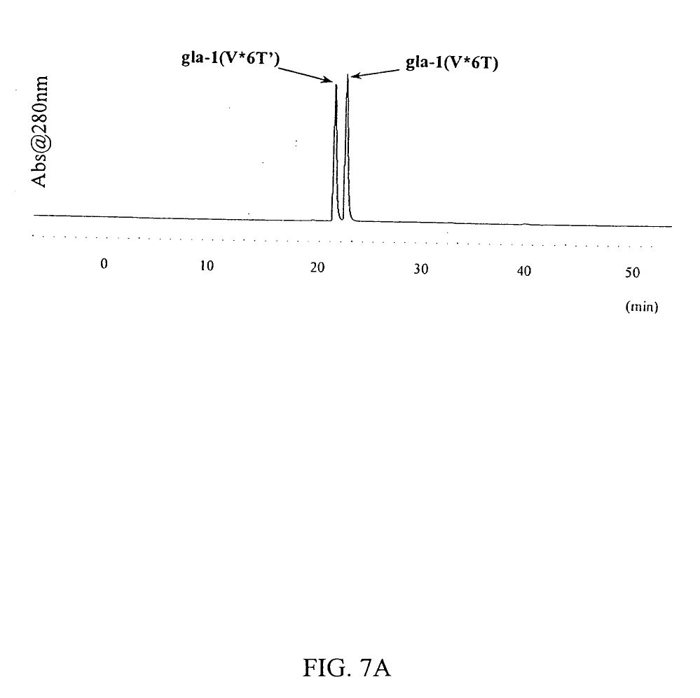

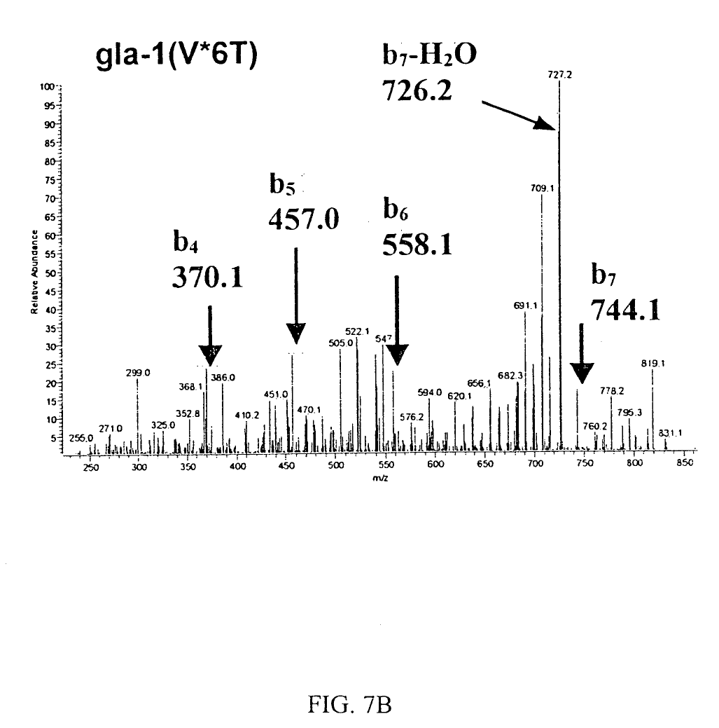

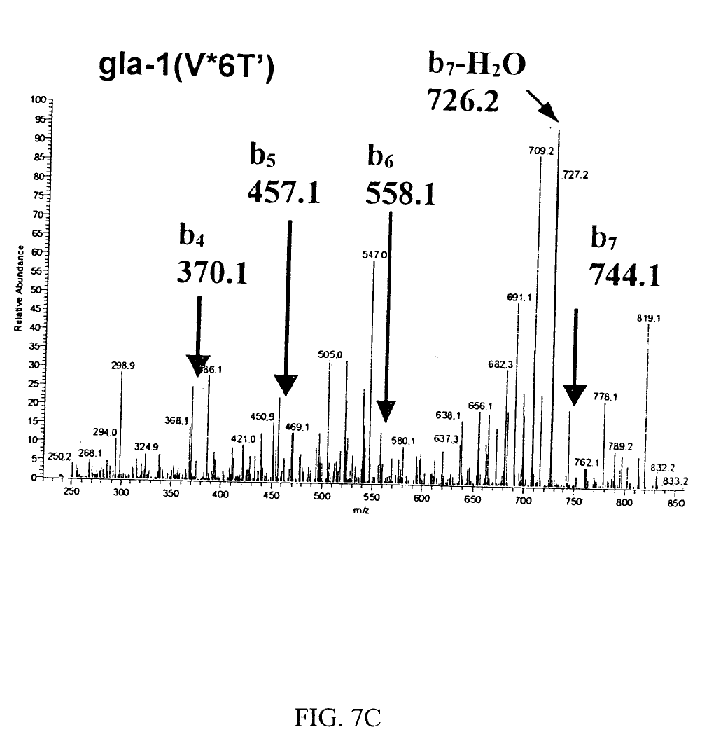

To establish the absolute configuration around the β carbon (C-3) in Hyv of the gla conopeptides, two diastereomeric analogs of gla-1, epimeric at C-3, were synthesized. These synthetic analogs were termed gla-1 (Hyv6Thr) and gla-1 (Hyv6Thr′), where Thr′ is allo-threonine. FIG. 7A shows the RP-HPLC chromatogram of the gla-1 (Hyv6Thr) and gla-1(Hyv6Thr′) analogs FIGS. 7B and 7C show the respective MS/MS spectral data for the gla-1 (Hyv6Thr) and gla-1 (Hyv6Thr′) analogs. Direct comparison of the chromatographic and spectrometric behavior of these analogs with known configurations at the chiral centers in residue 6 indicated that gla-1 is likely to correspond to the 2S, 3S configuration in its Hyv residue, whereas gla-1′ corresponds to the 2S, 3R configuration in its Hyv residue. Thus the results with the synthetic peptides of known chirality in residue 6 (Hyv) established that gla-1 and gla-1′ are diastereomeric at Hyv.

### Example 12

### Chemical Structure of γ-Hydroxyconophans

From the combined NMR, ESI-MS/MS, and Edman degradation analyses of the gla and mus octa-conopeptides, the following sequences were determined for these peptides:

| | | |
|---|---|---|
| gla-1/gla-1′ | A-O-A-N-S-V*-W-S | (SEQ ID NO: 5) |
| gla-2 | A-O-A-N-S-V-W-S | (SEQ ID NO: 7) |
| mus-1/mus-1′ | A-O-S-N-S-V*-W-S | (SEQ ID NO: 6) |
| mus-2 | A-O-S-N-S-V-W-S | (SEQ ID NO: 8) |

wherein:
O = γ-hydroxyproline;
V* = γ-hydroxyvaline; and
W = D-Trp.

As can be seen from a comparison of SEQ ID NOS:5-8, the gla conophans differ from their mus counterparts at residue 3, by the presence of Ala, as opposed to Ser. Accordingly, a generalized structure for the gla and mus family of octapeptide conopeptides is the following: A-O-X₁-N-S-X₂-W-S (SEQ ID NO:1)

wherein:

O is γ-hydroxyproline;

X₁ is A or S;

X₂ is V or γ-hydroxyvaline (V*); and

W is D-Tryptophan.

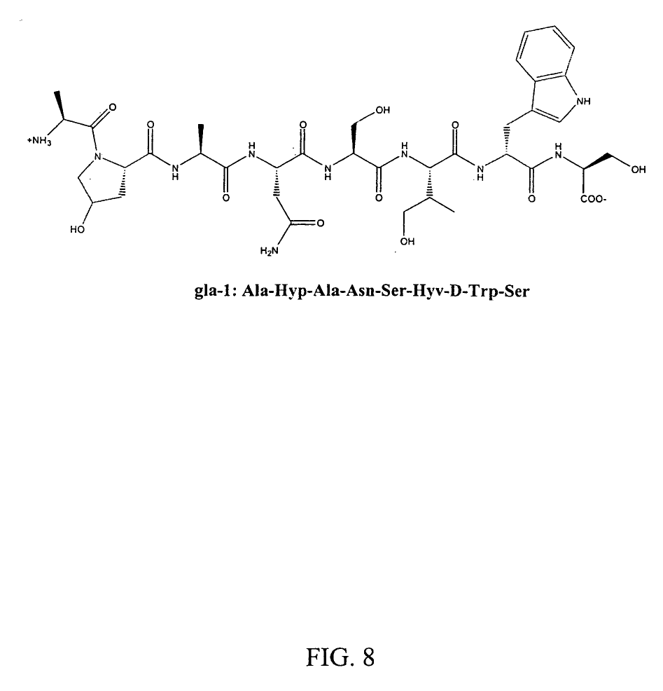

A preferred embodiment of a γ-Hydroxyconophan octapeptide is gla-1 (SEQ ID NO:5), which has the chemical structure shown in FIG. 8.

### Example 13

### Molecular Modeling of Hydroxyconophan Structure

Molecular models were built and optimized using the MMX force-field as described (Mari, F., Lahti, P. M., McEwen, W. E. Journal of the American Chemical Society 1992, 114, 813-821). The structural motif that characterizes the gla/mus γ-Hydroxyconophan family of conopeptides [Hyv-D-Trp] was capped using an acetyl group at the N-terminus and an amide group at the C-terminus to simulate a protein-like environment. An extended conformation consistent with the NMR data was used for the initial model and optimized to self-consistency by the force field.

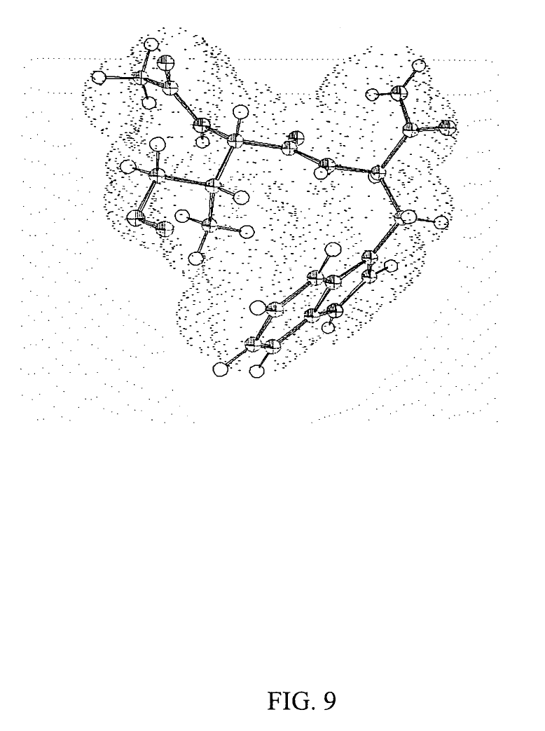

FIG. 9 shows a molecular model of a γ-Hydroxyconophan including the structural motif H₃CC(O)-Hyv-D-Tp-NH₂. This model is consistent with the stabilizing interactions and NMR data described above. It is known that intramolecular lactone formation cannot occur when constrained hydroxylated amino acids such as Hydroxyproline (Hyp) are found in polypeptide chains (Jimenez, E. C., Olivera, B. M., Gray, W. R., Cruz, L. J. Journal of Biological Chemistry 1996, 271, 28002-28005; Jacobsen, R., Jimenez, E. C., Grilley, M., Watkins, M., Hillyard, D., Cruz, L. J., Olivera, B. M. Journal of Peptide Research 1998, 51, 173-179; Pallaghy, P. K., Melnikova, A. P., Jimenez, E. C., Olivera, B. M., Norton, R S. Biochemistry 1999, 38, 11553-11559; Raybaudi Massilia, G., Eliseo, T., Grolleau, F., Lapied, B., Barbier, J, Bournaud, R., Molgo, J., Cicero, D. O., Paci, M., Eugenia Schinina, M., Ascenzi, P., Polticelli, F. Biochemical and Biophysical Research Communications 2003, 303, 238-246). The model indicates that the D-configuration of the Trp residue is required to disfavor the intraresidue cleavage of the peptide bond by the γ-hydroxyl group.

### Example 14

### Neuronal Cell Culture System for Testing In Vitro Biological Activity of Conopeptides

Primary cultures of cortex were prepared from cortical hemispheres isolated from anaesthetized neonatal rat pups. Hemispheres were cleaned of meninges and the hippocampus removed and discarded. The cortex was dissociated using 20 U/ml Papain and trypsin with constant mixing for 45 min at 37° C. Digestion was terminated with fraction V BSA (1.5 mg/ml) and Trypsin inhibitor (1.5 mg/ml) in 10 mls media (DMEM/F12+10% fetal Bovine serum and B27 neuronal supplement (Life Technologies). Using gentle trituration, cells were separated from the surrounding connective tissue. Using a fluid-handling robot (Quadra 96, Tomtec) cells were settled onto Poly-D-lysine coated white/clear 96 well plates (Becton-Dickenson). Each well was plated with approximately 25,000 cells. The plates were placed into a humidified 5% CO₂ incubator at 37° C. and kept for at least 7 days before fluorescence screening.

### Example 15

### Fluo-3 Assay of Cellular Ca⁺⁺ Flux in Cultured Cortical Neurons

The ability of γ-Hydroxyconophan compounds to modulate flux of Ca⁺⁺ ions in cultured neuronal cells was tested using a 96 well plate fluorimetry protocol utilizing the the Fluo-3 ester calcium-dye method. As is well known, this dye becomes effectively trapped within cells following cleavage of the ester from the Fluo-3 dye by esterases within the cell cytoplasm. Cells were incubated for 45 min at 37° C. The Fluo-3 dye (2 μM) was loaded into cells with 20% pluronic acid.

Prior to beginning the experiments, the cells were washed thoroughly with saline solution using a Tomtec liquid handling robot. Initial readings were taken with a cytofluor 4000 HT fluorimeter (Perceptives) set at 36° C. to assess the background fluorescence in each of the plates. The excitation/emission settings were 483/530.

Test compounds were added with a liquid handling robot as described above. All compounds were tested at a concentration 3 μM. Some compounds were screened in both undepolarized and depolarized (10 mM KCl) conditions. For those experiments that required pretreatment, cells were pretreated for 30s with the compound before application of 10 mM KCl. This concentration of KCl was selected based on the results of a KCl concentration-response experiment performed in primary cultures of rat neonatal cortex which demonstrated that a significant and robust increase in intracellular calcium was observed at this concentration. In each plate, a control saline well was included and either a second saline well or a KCl well were included. The saline solution used in the assays contained (in mM) 137 NaCl, 5 KCl, 10 HEPES, 25 Glucose, 3 CaCl₂, and 1 MgCl₂ (brought to pH 7.3 with NaOH). Each compound was run in 4 wells per plate in 3 separate plates. Readings were taken at intervals of 15s, 2 min, 5 min and 10 min.

Data were analyzed using Microsoft Excel and graphed using Origin software (Microcal). The initial background fluorescent readings were subtracted from the treatment readings. The saline control was then subtracted and the results expressed as an absolute change in fluorescence. Increases in intracellular calcium measured with the Fluo-3 dye are reflected as rises in fluorescence. Conversely, decreases in fluorescence represent a drop in intracellular calcium.

### Example 16

### Modulation of Neuronal Ca⁺⁺ Flux By Conopeptides

Several γ-Hydroxyconophan and Conophan compounds were tested in the assay described in Example 16 for their ability to modulate cellular Ca⁺⁺ flux in cultured neurons. The results showed that gla-1, gla-1′ and gla-2 at 3 μM concentration all had the effect of reducing Ca⁺⁺ flux in the cells. By contrast, the saline control had the opposite effect, causing an increase in cellular Ca⁺⁺. This result demonstrated that the γ-Hydroxyconophan and Conophan peptides tested were biologically active, modulating the flux of calcium in the cells, presumably through interaction with cell surface receptors controlling calcium levels in the cells.

### Other Embodiments

This description has been by way of example of how the compositions and methods of the invention can be made and carried out. Various details may be modified in arriving at the other detailed embodiments, and many of these embodiments will come within the scope of the invention. Therefore, to apprise the public of the scope of the invention and the embodiments covered by the invention, the following claims are made.

What is claimed is:

1. An isolated γ-Hydroxyconophan peptide, said peptide comprising the amino acids γ-OH-Val and D-Trp in contiguous residues.

2. An isolated peptide comprising the amino acid sequence of SEQ ID NO:2.

3. An isolated peptide comprising the amino acid sequence of SEQ ID NO:3.

4. An isolated peptide comprising the amino acid sequence A-O-X₁-N-S-X₂-W-S (SEQ ID NO:1) wherein:
O is γ-hydroxyproline;
X₁ is A or S;
X₂ is V or γ-hydroxyvaline (V*); and
W is D-Tryptophan.

5. The peptide of claim 4, having the amino acid sequence of SEQ ID NO:5.

6. The peptide of claim 4, having of the amino acid sequence of SEQ ID NO:6.

7. The peptide of claim 4, having the amino acid sequence of SEQ ID NO:7.

8. The peptide of claim 4, having the amino acid sequence of SEQ ID NO:8.

9. The peptide of claim 1, isolated from an animal.

10. The peptide of claim 1, wherein said peptide is synthesized by man.

11. A composition comprising the isolated peptide of claim 1, in a pharmaceutically acceptable carrier.

12. A composition comprising the isolated peptide of claim 4, in a pharmaceutically acceptable carrier.

13. A method of modulating the level of an ion within a cell, the method comprising the steps of:
(a) providing a cell that responds to a peptide that binds to a chemical structure on the surface of said cell by modulating the level of at least one ion within said cell; and
(b) contacting said chemical structure on the surface of said cell with a peptide comprising the amino acids γ-OH-Val and D-Trp in contiguous residues, wherein said peptide selectively binds to said chemical structure.

14. The method of claim 13, wherein the chemical structure on the surface of said cell is a cell surface receptor.

15. The method of claim 14, wherein said receptor is of a receptor type selected from a calcium channel, a sodium channel, a potassium channel, and a chloride channel.

16. The method of claim 15, wherein said receptor is voltage-gated.

17. The method of claim 13, wherein said peptide comprises the backbone amino acid sequence of SEQ ID NO:2 or SEQ ID NO:3.

18. The method of claim 13, wherein said ion is at least one selected from the group consisting of a calcium ion, a sodium ion, a potassium ion and a chloride ion.

19. A method of modulating the level of an ion within a cell, the method comprising the steps of:
(a) providing a cell that responds to a peptide that binds to a chemical structure on the surface of said cell by modulating the level of at least one ion within said cell; and
(b) contacting said chemical structure on the surface of said cell with a peptide comprising the amino acids A-O-X₁-N-S-X₂-W-S (SEQ ID NO:1) in contiguous residues, wherein said peptide selectively binds to said chemical structure, wherein:
O is γ-hydroxyproline;
X₁ is A or S;
X₂ is V or γ-hydroxyvaline (V*); and
W is D-Tryptophan.

20. The method of claim 19, wherein said peptide has an amino acid sequence selected from the group consisting of SEQ ID NOS:5, 6, 7, and 8.


SEQUENCE LISTING (8 sequences)

SEQ ID NO: 1 - protein, 8 aa, Unknown Organism
  Description of Unknown Organism Illustrative peptide formula sequence
APXNSXWS

SEQ ID NO: 2 - protein, 8 aa, Conus gladiator
APANSVWS

SEQ ID NO: 3 - protein, 8 aa, Conus mus
APSNSVWS

SEQ ID NO: 4 - protein, 8 aa, Unknown Organism
  Description of Unknown Organism Illustrative peptide formula sequence
APXNSVWS

SEQ ID NO: 5 - protein, 8 aa, Conus gladiator
  MOD_RES (2): gamma-hydroxyproline
APANSVWS

SEQ ID NO: 6 - protein, 8 aa, Conus mus
  MOD_RES (2): gamma-hydroxyproline
APSNSVWS

SEQ ID NO: 7 - protein, 8 aa, Conus sp.
  MOD_RES (2): gamma-hydroxyproline
APANSVWS

SEQ ID NO: 8 - protein, 8 aa, Conus sp.
  MOD_RES (2): gamma-hydroxyproline
APSNSVWS